(19) United States
(12) Patent Application Publication
(10) Pub. No.: US 2004/0033276 A1
(43) Pub. Date: Feb. 19, 2004

(54) Coating composition for cereals having a preventive or treating effect on diabetes and cereals coated thereby

(76) Inventors: Jeong-Chan Ra, Suwon-si (KR); Kyung-Sun Kang, Seoul (KR); Jin-Hee Bae, Incheon (KR); Dow Kwon, Cheonan-si (KR)

Correspondence Address: David A. Einhorn, Esq., Anderson Kill & Olick, P.C., 1251 Avenue of the Americas, New York, NY, 10020, US

(21) Appl. No.: 10/632,266

(22) Filed: Jul. 29, 2003

(30) Foreign Application Priority Data
Jul. 27, 2002 (KR) 10-2002-0044440

(51) Int. Cl. A61K 35/78; A61K 47/00

(52) U.S. Cl. 424/728; 424/774; 424/777; 424/750; 424/439

(57) ABSTRACT

A composition for coating cereals comprising an extract of mulberry leaves(Mori Folium) or an extract of a herb mixture comprising mulberry leaves and a starch solution having a concentration ranging from 10 to 50 % (w/v), the extract:starch solution weight ratio being in the range of 21:1 to 21:10, wherein the herb mixture consists of mulberry leaves and a herb medicine selected from the group consisting of Panax Ginseng, Mori Fructus, Lycil Fructus and a mixture thereof.

### FIELD OF THE INVENTION

The present invention relates to a composition for coating cereals having a preventive or treating effect on diabetes and cereals coated thereby.

### BACKGROUND OF THE INVENTION

Diabetes is one of the incurable diseases and currently available therapeutic methods therefor include diet and movement cures in combination with a drug treatment. In Korea, a folk remedy employing natural materials such as herb medicines is widely used. However, the effect of those materials used in the folk remedy is generally unsupported scientifically and they may have problems of toxicity due to excess intake and contamination or degeneration during the processing and storage.

Meanwhile, it has been reported that the mulberry leaf(Mori Folium) lowers the blood glucose level in model mice suffering from diabetes induced by the administration of streptozotocin(Chen, F. J., et al, Yakugaki Zasshi, 115: 476-482(1995)), and exerts such activity through the inhibition of the polysaccharide digestive enzyme in the intestines(lee J. S. et al., Yakhak Hoeji, 39(4): 367-372(1995)).

Further, Chung S. H. et al. reported that mulberry leaf contains plentiful myoinositol which is known to have a treating effect on the diabetic neuropathy(The KyungHee J. of Genet & Mol. Biol., 8: 38-44(1996)),.

Other materials known to be effective for treating diabetes include Mori Fructus, Lycil Fructus, Panax Ginseng and the others.

Mori Fructus has been reported to lower the blood glucose level in a model mouse for insulin-independent diabetes established by the administration of streptozotocin(Kim, T. W., et al, J. Oriental Pharmacy, 38(2): 100-107(1996)).

Lycil Fructus is known to be effective for hypertension, hyperlipidemia and hyperglucocemia(Kim, N. J. et al., Kor J. Pharmacogn., 25(3): 263-271(1994)).

Panax Ginseng has been reported to recuperate the internal organs, stabilize the spirit, alleviate the swelling, and have treating activities for dizziness, headache, frequent urination, hematemesis and apoplexy(Oh, J. S., Korean J. Ginseng Sci., 1(1): 1-12(1976)).

Generally, treatment of diabetes is accompanied with a diet cure, wherein daily intake of calorie is limited within the range of 25 to 50 kcal/kg body weight. The amount of polysaccharide permitted for a day may range from 150 to 300 g, which corresponds to 1 to 2 bowls of cooked rice. Accordingly, other cereals having low calories such as unpolished rice and barley are mixed with rice for lowering the calories. However, people, especially the aged, do not like such low-calorie cereals due to the inferior taste and digestibility.

Meanwhile, a variety of coated cereals have been prepared by coating cereals with various materials having beneficial effects. For instance, rice have been coated by chitosan(Korean Patent Publication No. 2001-44203), maltose(Korean Patent publication No. 2000-18757), gelatin(Korean Patent publication No. 2000-47012), etc. However, most of the coated cereals have the problem that the coating materials come off during boiling and steaming.

The present inventors have endeavored to develop improved coated cereals which have a preventive or treating effect on diabetes.

### SUMMARY OF THE INVENTION

Accordingly, it is an object of the present invention to provide a composition for coating cereals, which have a preventive or treating effect on diabetes and the coating thereof does not come off during cooking.

Another object of the present invention is to provide coated cereals prepared by coating cereal with said composition.

A further object of the present invention is to provide a method for preparing said coated cereals.

In accordance with one aspect of the present invention, there is provided a composition for coating cereals comprising an extract of mulberry leaves(Mori Folium) or an extract of a herb mixture comprising mulberry leaves and a starch solution having a concentration ranging from 10 to 50 % (w/v), the extract:starch solution weight ratio being in the range of 21:1 to 21:10, wherein the herb mixture consists of mulberry leaves and a herb medicine selected from the group consisting of Panax Ginseng, Mori Fructus, Lycil Fructus and a mixture thereof.

In accordance with another aspect of the present invention, there are provided coated cereals having a blood glucose level-lowering effect, which is prepared by coating cereals with the inventive composition.

In accordance with a further aspect of the present invention, there is provided a process for preparing the inventive coated cereal, comprising the steps of mixing the inventive composition and cereals in a ratio ranging from 10 g to 100 g of composition/kg cereal to coat the cereal with the composition and drying the coated cereal.

### DETAILED DESCRIPTION OF THE INVENTION

The inventive composition for coating cereals comprises an extract of mulberry leaves as an active ingredient for exhibiting preventive or treating effect on diabetes by lowering blood glucose level.

The inventive composition may further comprise, as an active ingredient, an extract of a herb medicine selected from the group consisting of Panax Ginseng, Mori Fructus, Lycil Fructus and a mixture thereof, for a synergistic effect. In case of a composition comprising extracts of mulberry leaves and Panax Ginseng, the weight ratio of the mulberry leaves and Panax Ginseng ranges from 5:1 to 20:1, wherein 10:1 is preferred. The inventive composition may comprise extracts of mulberry leaves, Panax Ginseng and a herb medicine selected from the group consisting of Mori Fructus, Lycil Fructus and a mixture thereof in a weight ratio ranging from 10:2:5 to 20:1:8. A composition comprising extracts of mulberry leaves, Panax Ginseng, Mori Fructus and Lycil Fructus in a weight ratio of 2:0.2:2:1 is most preferred due to its highest activity for lowering blood glucose level.

An extract of mulberry leaves or a herb mixture including same may be prepared by a process comprising the steps of: pulverizing mulberry leaves or the herb mixture to powder having a particle size ranging from 30 to 50 meshes; extracting the powder with one to three-fold volume of water or spirit(concentration: 40 to 80%) for a period ranging from 48 to 72 hours; filtering the resulting extract with a filter having a pore size ranging from 10 to 100 μm; concentrating the filtrate by 2 to 6 folds under a reduced pressure at a temperature ranging from 60 to 80° C. for a period ranging from 24 to 60 hours; and cooling the resulting concentrate to a temperature below 30° C.

Otherwise, an extract of a herb mixture may be prepared by extracting respective herb medicine in accordance with the method as above and mixing the resulting extracts at a given ratio.

For the purpose of the present invention, preferred is an extract having a glucose concentration ranging from 15 to 30 brix %, specific gravity ranging from 1.00 to 1.20 and refractive index ranging from 1.30 to 1.40.

The inventive cereal-coating composition comprises a starch solution having a concentration of 10 to 50% in order to allow the composition to expand simultaneously with the expansion of the cereals during cooking, thereby preventing the peeling off and dispersion of the coating on cereals.

Exemplary starches for use in the inventive composition include starches from rice, potato, corn and sweet potato.

The inventive composition may comprise the extract of mulberry leaves or an extract of a herb mixture comprising same and a starch solution in a weight ratio ranging from 21:1 to 21:10, preferably, 21:3 to 21:5.

The cereals that may be coated with the inventive composition may be selected from the group consisting of polished rice, unpolished rice, barley, millet, German millet, African millet and the like cereals.

The present invention also provides coated cereals which are prepared by a process comprising the steps of mixing the inventive composition and cereals in a ratio ranging from 10 g to 100 g, preferable 20 to 30 g, of the composition/kg cereal to coat the cereal with the composition and drying the coated cereal at a temperature ranging from 30 to 60° C. for a period ranging from 30 min. to 2 hours.

The inventive coated cereals exhibit a blood glucose level-lowering effect and a preventive or treating effect for diabetes. Further, the inventive coated cereals are advantageous in that the problem of dispersion of coating material does not occur during cooking and they have superior qualities such as appearance, scent and taste to uncoated cereals in sensuous tests. Accordingly, the inventive coated cereal can be advantageously used as a functional food in a diet cure for preventing or treating diabetes.

The following Examples are intended to further illustrate the present invention without limiting its scope.

Further, percentages given below for solid in solid mixture, liquid in liquid, and solid in liquid are on a wt/wt, vol/vol and wt/vol basis, respectively, and all the reactions were carried out at room temperature, unless specifically indicated otherwise.

### REFERENCE EXAMPLE 1

Preparation of a Mouse Model for Diabetes

Six week-old male mice weighing 25 to 30 g were administered intravenously with 120 mg/kg of alloxan and, those mice of which blood glucose level was over 200 mg/dl were selected and acclimated for one week.

### REFERENCE EXAMPLE 2

Preparation of an Extract

Each of test dried herb medicines was pulverized to a powder having a particle size ranging from 30 to 50 mesh and extracted with an equal volume of 70% spirit(ethanol) for 72 hours. The resulting extract was filtered through a 0.1 μm filter (Satorius, Germany), and the filtrate was concentrated under a reduced pressure at 80° C. for 48 hours and cooled to below 30° C.

### EXAMPLE 1

Blood Glucose Level-lowering Activities of Extracts Containing Mulberry Leaves and Panax Ginseng in Various Ratios

Diabetes model mice prepared in Reference Example 2 were divided into five groups each consisting of 8 mice. Four of the five groups of mice were orally administered for 7 days with 250 g/kg of each of the extracts having the composition listed in Table 1 and prepared by the method of Reference Example 2, and the remaining one group of mice were used as a control group. The mice were starved for 18 hours and their blood glucose levels were measured.

The blood glucose levels of mice were measured before and after the administration of the herb extract and changes in the levels are shown in Table 1.

TABLE 1

| | Blood glucose level (mg/dl) | | Change in |
|---|---|---|---|
| | Before | After administration | blood glucose |
| Group (n = 8) | administration | for 7 days | level (mg/dl) |
| Control | 472.9 ± 10.1 | 486.8 ± 17.0 | +2.94 |
| Mulberry leaves | 477.3 ± 10.8 | 445.6 ± 14.8 | −6.64 |
| Mulberry leaves + | 475.8 ± 6.5 | 387.6 ± 22.2 | −18.54 |
| Panax Ginseng | | | |
| (10:1(w/w)) | | | |
| Mulberry leaves + | 477.6 ± 8.0 | 432.5 ± 23.2 | −9.44 |
| Panax Ginseng | | | |
| (15:1(w/w)) | | | |
| Mulberry leaves + | 478.1 ± 9.9 | 426.0 ± 28.8 | −10.90 |
| Panax Ginseng | | | |
| (20:1(w/w)) | | | |

As can be seen from Table 1, the experimental groups show significantly lowered blood glucose levels, the best result being observed for the experimental group administered with Mulberry leaves+Panax Ginseng (10:1(w/w)).

### EXAMPLE 2

Blood Glucose Level-lowering Activities of Extracts Having Various Compositions

Added to the mixture of Mulberry leaves and Panax Ginseng (10:1(w/w))(“mulberry leaves/Panax Ginseng”), which showed the highest activity in the test of Example 1, were Mori Fructus and Lycil Fructus in various weight ratios as listed in Table 2. The resulting herb mixtures were extracted by the method of Reference Example 2 and blood glucose level-lowering activities of the resulting extracts were measured in accordance with the method of Example 1. Mice of the control group were not administered with any herb extract. The result is shown in Table 2.

TABLE 2

| | Blood glucose level (mg/dl) | |
|---|---|---|
| | Before | After administra- |
| Group (n = 8) | administration | tion for 7 days |
| Control | 434.4 ± 14.4 | 435.9 ± 18.7 |
| Mulberry leaves/Panax Ginseng + | 432.1 ± 9.4 | 400.5 ± 13.8 |
| Mori Fructus + Lycil Fructus (1:1:1) | | |
| Mulberry leaves/Panax Ginseng + | 430.0 ± 4.9 | 395.6 ± 12.0 |
| Mori Fructus + Lycil Fructus (2:1:1) | | |
| Mulberry leaves/Panax Ginseng + | 436.0 ± 9.2 | 376.4 ± 11.9 |
| Mori Fructus + Lycil Fructus (2:2:1) | | |

As can be seen from Table 2, the experimental groups show significantly lowered blood glucose levels, the best result being observed for the experimental group administered with an extract of a herb mixture consisting of Mulberry leaves/Panax Ginseng+Mori Fructus+Lycil Fructus (2:2:1(w/w/w)).

### EXAMPLE 3

Changes in Diabetic Parameters by Administration of Herb Extracts

Herb extracts were prepared by the method of Reference Example 2 from a herb mixture consisting of mulberry leaves+Mori Fructus+Lycil Fructus (2:2:1 (w/w/w))(“Extract A”) and a herb mixture consisting of mulberry leaves+Mori Fructus+Lycil Fructus+Panax Ginseng (2:2:1:0.2(w/w/w/w)(“Extract B”), respectively. Each of the resulting extracts was administered orally by Sonde to a group of seven diabetic mice prepared as in Reference Example 1 at a dose of 0.5 ml/day for 4 weeks. The resulting diabetic mice were starved for 18 hours and, then, examined as in Table 3. The control group consisted of seven normal healthy mice and the diabetes group consisted of seven diabetic mice induced with alloxan and administered with no herb mixture.

TABLE 3

| | Group | | | |
|---|---|---|---|---|
| | | | Extract | |
| | Control group | Diabetes group | A | Extract B |
| Glucose | 115.3 ± 4.39 | 216.1 ± 12.08 | 94.9 ± | 89.4 ± 4.96 |
| (mg/dl) | | | 5.94 | |
| Urea (mg/dl) | 35.9 ± 2.12 | 53.2 ± 3.63 | 38.2 ± | 43.7 ± 3.79 |
| | | | 3.41 | |
| Creatinine | 0.26 ± 0.02 | 0.61 ± 0.05 | 0.42 ± | 0.43 ± 0.02 |
| (mg/dl) | | | 0.02 | |
| Total protein | 5.43 ± 0.11 | 4.1 ± 0.17 | 5.7 ± | 5.2 ± 0.21 |
| (g/dl) | | | 0.25 | |
| Albumin(A) | 3.9 ± 0.292 | 2.1 ± 0.079 | 3.4 ± | 3.4 ± 0.169 |
| (g/dl) | | | 0.182 | |
| Globulin(G) | 1.82 ± 0.213 | 1.99 ± 0.149 | 2.01 ± | 1.68 ± 0.195 |
| (g/dl) | | | 0.076 | |
| A/G ratio | 2.75 ± 0.162 | 1.17 ± 0.086 | 1.86 ± | 2.33 ± 0.105 |
| | | | 0.079 | |
| Billirubin | 7.2 ± 0.24 | 10.3 ± 0.61 | 7.7 ± | 8.1 ± 0.27 |
| (mg/dl) | | | 0.26 | |

As can be seen from Table 3, the blood glucose level of the diabetes group increased by about 93% in comparison with that of the control group, suggesting that diabetes was induced by the administration of alloxan. Further, blood glucose levels of the Extract A- and Extract B-administered groups decreased by 57% and 59%, respectively, in comparison with that of the diabetes group, suggesting that the active components of saponins, alkaloids and tannins in Extracts A and B prevent the increase of blood glucose level. Increases in the levels of urea and creatinine in the blood reflect the renal dysfunction, and the diabetes group exhibited increased levels of blood urea and creatinine by 49% and 138%, respectively, in comparison with those of the control group. In stark contrast, blood urea levels of the Extract A- and Extract B-administered groups increased by 26% and 15%, respectively, and blood creatinine levels of the Extract A- and Extract B-administered groups, 31% and 28%, respectively. Moreover, decreases in the concentrations of total proteins and albumin in blood, which are important clinical markers of protein catabolism or nephropathy accompanying diabetes, were observed in the diabetes group, while the Extract A- and Extract B-administered groups show levels which were close to those of the control group.

### EXAMPLE 4

Inhibition of α-glucosidase by Extracts of Herb Medicine

Inhibitory activities of various herb extracts on the activity of α-glucosidase, which catalyzes the digestion of a polysaccharide to monosaccharides, were examined as follows.

Extracts of various herb medicines listed in Table 4 below were prepared in accordance with the method of Reference Example 2.

Added to 0.27 ml of 2 mM substrate (p-nitrophenyl-α-D-glucopyranoside, Sigma, U.S.A.) were 0.03 ml of α-glucosidase(Sigma, U.S.A.), 0.2 ml of 20 mM phosphate buffer (pH 7.0) and 0.1 ml of respective extracts obtained above. The resulting mixture was incubated in a 37° C. thermostat(Vision Science, Korea) for 30 minutes, and 0.5 ml of 1 M glycine-NaOH (pH 9.0) was added thereto to stop the reaction. The reaction mixture was centrifuged at 3,000 rpm for 10 minutes and 0.2 ml of the resulting supernatant was added to the wells of a 96-well microtiter plate (Nunc, Denmark) and the absorbance at 405 nm was read with ELISA Reader (SUNRISE TECAN, Austria).

Negative control was treated with distilled water instead of the herb extract, and positive control, with acarbose(Glucobay™, Bayer) and DNJ (Deoxynojirimycin, Dep. Seruculture & Entomology, NIAST), which are representative α-glucosidase inhibitors.

Activities of the respective extracts for inhibiting α-glucosidase were calculated by the following equation and the result is shown in Table 4.

Inhibition (%)=(1−Absorbance of a sample/Absorbance of the negative control)×100

TABLE 4

| Herb medicine | Absorbance | Inhibition (%) |
|---|---|---|
| Negative Control | 3.9773 ± 0.0104403 | 0 |
| Positive Control 1 (Acabose) | 1.5033 ± 0.0051316 | 62.2 |
| Positive Control 2 (DNJ) | 0.7641 ± 0.0021 | 80.79 |
| Mulberry leaves | 1.4982 ± 0.01253 | 62.33 |
| Mori Fructus | 1.6213 ± 0.0198578 | 59.24 |
| Lycil Fructus | 2.8903 ± 0.0282184 | 27.33 |
| Panax Ginseng | 3.6777 ± 0.0763042 | 7.53 |
| Mulberry leaves + Mori Fructus | 0.8321 ± 0.0765871 | 79.08 |
| Mulberry leaves + Lycil Fructus | 1.0327 ± 0.2852148 | 74.04 |
| Mulberry leaves + Mori Fructus + | 0.6012 ± 0.0874590 | 84.88 |
| Lycil Fructus | | |
| Mulberry leaves + Schizandra | 2.0903 ± 0.2821846 | 47.44 |
| Frunctus | | |
| Mulberry leaves + Liriopis Tuber | 1.5742 ± 0.0004560 | 60.42 |

As can be seen from Table 4, all of the extracts of herb mixtures containing mulberry leaves exhibited extents of inhibition of over 60%, the highest inhibitory activity being observed for the mixture of mulberry leaves, Mori Fructus and Lycil Fructus.

### EXAMPLE 5

Preparation of Coating Composition and Diffusion Test

Polished rice was ground to 80 to 150 mesh, added to various amounts of water and the mixtures were heated with stirring until fully glutenized, to obtain starch solutions having concentrations ranging from 5 to 50%. An extract of a herb mixture consisting of mulberry leaves, Mori Fructus, Lycil Fructus and Panax Ginseng (2:2:1:0.2(w/w/w/w)) prepared as in Reference Example 2 was mixed with the respective starch solutions in a weight ratio of 21:4 to obtain coating compositions.

25 g of the coating composition was added to 1 kg of rice and the mixture was mixed thoroughly with a mixer(RNL Lifescience Co., Ltd) to obtain rice coated with the composition. When a coating machine(RNL Lifescience Co., Ltd) was used instead of the mixer, the composition was sprayed with a spray gun onto rice. The resulting coated rice was dried at 50° C.

Degrees of diffusion of the coating compositions upon cooking were examined as follows.

Washed polished rice was added to a kettle having a diameter of 20 cm and the coated rice prepared as above was added to a well (size: 10 cm id×4 cm height) positioned in the center of the rice layer. The rice was boiled until glutenized and observed to determine the extent of difflusion of the coating composition. As the coating composition was darkish, the extent of diffusion of the coating composition was determined by observing the spread of color.

The result is shown in Table 5.

TABLE 5

| | | | | | | | |
|---|---|---|---|---|---|---|---|
| Conc. of starch | 5% | 10% | 15% | 20% | 30% | 40% | 50% |
| solution | | | | | | | |
| Diffusion | Yes | No | No | No | No | No | No |

As can be seen from Table 5, the difflusion of the coating composition did not occur when the concentration of the starch solution employed therein was 10% or more. However, when starch solutions having concentrations of over 50% were used, the rice became hard and the feeling of chewing became bad. Accordingly, it is preferable to use a starch solution having a concentration ranging from 10 to 50%.

### EXAMPLE 6

Sensuous Tests

Sensuous tests on the polished rice and the inventive coated rice were carried out by a panel of 40 persons in their twenties to fifties, as follows.

Boiled rice was prepared by employing the rice coated with a composition containing an extract of a herb mixture consisting of mulberry leaves, Mori Fructus, Lycil Fructus and Panax Ginseng (2:2:1:0.2(w/wlw/w)) and 10% starch solution, in a weight ratio of 21:4, and the quality of the coated rice was assessed by the panel as in Table 6. Each test item was scored in a scale of 0 to 10, boiled rice prepared with uncoated rice being given a score of 5. The result is shown in Table 6.

TABLE 6

| Diabetes patients | | | | | | | | Normal healthy people | | | | | | | |
|---|---|---|---|---|---|---|---|---|---|---|---|---|---|---|---|
| No. | Sex | Age | Appearance | Scent | Taste | Texture | Preference | No. | Sex | Age | Appearance | Scent | Taste | Texture | Preference |
| 1 | M | 42 | 5 | 6 | 5 | 5 | 6 | 1 | M | 29 | 4 | 3 | 5 | 5 | 6 |
| 2 | M | 39 | 6 | 6 | 6 | 4 | 6 | 2 | M | 31 | 6 | 5 | 5 | 6 | 6 |
| 3 | M | 50 | 5 | 5 | 8 | 5 | 7 | 3 | M | 35 | 5 | 5 | 6 | 6 | 6 |
| 4 | M | 51 | 5 | 5 | 5 | 5 7 | 4 | M | 40 | 6 | 6 | 5 | 6 | 7 | |
| 5 | M | 48 | 5 | 5 | 6 | 5 7 | 5 | M | 33 | 7 | 6 5 | 6 | 6 | | |
| 6 | M 46 | 7 | 6 | 5 | 5 6 | 6 | M | 54 | 5 | 6 6 | 5 | 5 | | | |
| 7 | M 44 | 4 | 6 | 6 | 4 7 | 7 | M | 27 | 3 | 3 | 4 | 4 | 6 | | |
| 8 | M 40 | 5 | 6 | 7 | 4 | 7 | 8 | M | 46 | 5 | 5 | 5 | 5 | 5 | |
| 9 | M 38 | 4 | 6 | 8 | 6 | 6 | 9 | M | 39 | 5 | 5 | 6 | 6 | 6 | |
| 10 | M | 55 | 4 | 6 | 8 | 6 | 8 | 10 | M | 43 | 6 | 5 | 6 | 5 | 6 |
| 11 | F | 51 | 5 | 6 | 8 | 5 | 7 | 11 | F | 24 | 7 | 6 | 6 | 6 | 6 |
| 12 | F | 50 | 5 | 5 | 6 | 5 | 7 | 12 | F | 26 | 4 | 5 | 5 | 5 | 5 |
| 13 | F | 46 | 6 | 5 | 7 | 5 | 8 | 13 | F | 28 | 4 | 5 | 5 | 4 | 5 |
| 14 | F | 47 | 6 | 7 | 7 | 6 | 7 | 14 | F | 28 | 5 | 5 | 6 | 5 | 5 |
| 15 | F | 44 | 5 | 5 | 6 | 6 | 8 | 15 | F | 30 | 5 | 6 | 8 | 6 | 5 |
| 16 | F | 52 | 6 | 4 | 7 | 6 | 8 | 16 | F | 32 | 6 | 6 | 8 | 7 | 6 |
| 17 | F | 55 | 6 | 5 | 8 | 7 | 8 | 17 | F | 20 | 5 | 5 | 8 | 6 | 5 |
| 18 | F | 50 | 6 | 5 | 7 | 6 | 7 | 18 | F | 21 | 5 | 6 | 6 | 6 | 5 |
| 19 | F | 45 | 8 | 5 | 7 | 7 | 8 | 19 | F | 26 | 5 | 6 | 7 | 6 | 5 |
| 20 | F | 39 | 6 | 6 | 7 | 6 | 8 | 20 | F | 26 | 5 | 6 6 | 6 | 5 | |

As can be seen from Table 6, the inventive coated rice exhibited qualities superior to the uncoated rice and generally preferred by most of the diabetes patients and healthy persons.

### EXAMPLE 7

Blood Glucose Lowering Effect of the Inventive Coated Rice

Ten healthy persons were divided into two groups each consisting of 5 persons and provided with meals of boiled rice prepared from uncoated rice and the inventive coated rice of Example 6, respectively. Specifically, the test subjects were provided for 4 weeks with boiled rice prepared from 100 g of the test rice as breakfast and lunch, respectively.

Blood glucose levels of the persons were determined with Precision Q.I.L(MediSense, Inc.) before the test and after one, two, three and four weeks from the test. The result is shown in Table 7.

TABLE 7

| | Glucose (mg/dl) | | | |
|---|---|---|---|---|
| | Coated rice | | Uncoated rice | |
| | | 1 hour after | | 1 hour after |
| | Before meals | the meals | Before meals | the meals |
| Week 0 | 91 ± 10.1 | 152.2 ± 6.9 | 90 ± 10.2 | 150.4 ± 7.9 |
| Week 1 | 87.8 ± 4.5 | 158 ± 9.9 | 88.8 ± 8.8 | 159 ± 11.9 |
| Week 2 | 86.8 ± 6.8 | 153.4 ± 10.9 | 88.6 ± 7.4 | 156 ± 6.4 |
| Week 3 | 90.2 ± 3.6 | 144 ± 9.7 | 84.4 ± 4.8 | 152.2 ± 6.3 |
| Week 4 | 85.6 ± 6.2 | 128.8 ± 9.4 | 88.6 ± 7.4 | 156 ± 6.4 |

As can be seen from Table 7, the increase in the blood glucose level at one hour after the meals was significantly lower for the coated rice group in comparison with the uncoated rice group, in a time-dependent mode.

Accordingly, even though the blood glucose level shows significant individual variations, it is expected that the blood glucose level can be controlled constantly by taking meals prepared from the inventive coated cereals for more than 4 weeks.

While the invention has been described with respect to the above specific embodiments, it should be recognized that various modifications and changes may be made to the invention by those skilled in the art which also fall within the scope of the invention as defined by the appended claims.

What is claimed is:

1. A composition for coating cereals comprising an extract of mulberry leaves(Mori Folium) or an extract of a herb mixture comprising mulberry leaves and a starch solution having a concentration ranging from 10 to 50% (w/v), the extract:starch solution weight ratio being in the range of 21:1 to 21:10, wherein the herb mixture consists of mulberry leaves and a herb medicine selected from the group consisting of Panax Ginseng, Mori Fructus, Lycil Fructus and a mixture thereof.

2. The composition of claim 1, wherein the herb mixture comprises mulberry leaves and Panax Ginseng in a weight ratio ranging from 5:1 to 20:1.

3. The composition of claim 1, wherein the herb mixture comprises mulberry leaves, Panax Ginseng and a herb medicine selected from the group consisting of Mori Fructus, Lycil Fructus and a mixture thereof in a weight ratio ranging from 10:2:5 to 20:1:8.

4. The composition of claim 3, wherein the herb mixture comprises mulberry leaves, Panax Ginseng, Mori Fructus and Lycil Fructus in a weight ratio ranging from 2:0.2:2:1.

5. The composition of claim 1, wherein the starch is selected from the group consisting of rice, potato, corn and a mixture thereof.

6. A coated cereal having a blood glucose level-lowering effect, which is prepared by coating cereals with the composition of claim 1.

7. The coated cereal of claim 6, which has a preventive effect on diabetes.

8. The coated cereal of claim 6, wherein the cereals are selected from the group consisting of polished rice, unpolished rice, barley, wheat, millet, German millet and African millet.

9. A coated cereal having a blood glucose level-lowering effect, which is prepared by coating cereals with the composition of claim 2.

10. The coated cereal of claim 9, which has a preventive effect on diabetes.

11. The coated cereal of claim 9, wherein the cereals are selected from the group consisting of polished rice, unpolished rice, barley, wheat, millet, German millet and African millet.

12. A coated cereal having a blood glucose level-lowering effect, which is prepared by coating cereals with the composition of claim 3.

13. The coated cereal of claim 12, which has a preventive effect on diabetes.

14. The coated cereal of claim 12, wherein the cereals are selected from the group consisting of polished rice, unpolished rice, barley, wheat, millet, German millet and African millet.

15. A coated cereal having a blood glucose level-lowering effect, which is prepared by coating cereals with the composition of claim 4.

16. The coated cereal of claim 15, which has a preventive effect on diabetes.

17. The coated cereal of claim 15, wherein the cereals are selected from the group consisting of polished rice, unpolished rice, barley, wheat, millet, German millet and African millet.

18. A process for preparing a coated cereal having a blood glucose level-lowering effect, comprising the steps of mixing the composition of claim 1 and cereals in a ratio ranging from 10 to 100 g composition/kg cereal to coat the cereal with the composition and drying the coated cereal.

19. The process of claim 18, wherein the cereals are selected from the group consisting of polished rice, unpolished rice, barley, wheat, millet, German millet and African millet.

20. The process of claim 18, wherein the coated cereal is dried at a temperature ranging from 30 to 60° C.
